(19) United States
(12) Patent Application Publication
(10) Pub. No.: US 2014/0276203 A1
(43) Pub. Date: Sep. 18, 2014

(54) Biopsy Tool Having Micropattern

(71) Applicant: BOSTON SCIENTIFIC SCIMED, INC., Maple Grove, MN (US)

(72) Inventors: William Bertolino, Framingham, MA (US); Paul Smith, Smithfield, RI (US); James Weldon, Newton, MA (US)

(73) Assignee: BOSTON SCIENTIFIC SCIMED, INC., Maple Grove, MN (US)

(21) Appl. No.: 14/210,915

(22) Filed: Mar. 14, 2014

Related U.S. Application Data
(60) Provisional application No. 61/798,991, filed on Mar. 15, 2013.

(51) Int. Cl. A61B 10/06

(52) U.S. Cl. CPC A61B 10/06
USPC 600/564

(57) ABSTRACT

A biopsy forceps system including a biopsy forceps tool having a first jaw and a second jaw, wherein each of the first jaw and second jaw has a cutting edge and wherein a portion of at least one of the first jaw and the second jaw has a micropatterned polymer coating thereon.

### CROSS REFERENCE TO RELATED APPLICATIONS

This application claims the benefit of provisional U.S. Patent Application Ser. No. 61/798,991 (entitled BIOPSY TOOL HAVING MICROPATTERN, filed on Mar. 15, 2013), which is hereby incorporated by reference in its entirety.

The following patent applications are incorporated herein by reference, each in its entirety:

U.S. Pat. App. Ser. No. 61/798,685 (Firstenberg et al.), entitled ANTI-MIGRATION MICROPATTERNED STENT COATING, filed on Mar. 15, 2013 (Atty. Docket No. 563.2-15576U502);

U.S. Pat. App. Ser. No. 61/798,897 (Seddon et al.), entitled ANTI-MIGRATORY STENT COATING, filed on Mar. 15, 2013 (Atty. Docket No. S63.2-15705US01);

U.S. Pat. App. Ser. No. 61/798,794 (Clerc), entitled DELIVERY DEVICE FOR PARTIALLY UNCONSTRAINED ENDOPROSTHESIS, filed on Mar. 15, 2013 (Atty. Docket No. 563.2-15804US01);

U.S. Pat. App. Ser. No. 61/799,312 (Fleury et al.), entitled SUPERHYDROPHOBIC COATING FOR AIRWAY MUCUS PLUGGING PREVENTION, filed on Mar. 15, 2013 (Atty. Docket No. 563.2-15857US01); and

U.S. Pat. App. Ser. No. 61/798,545 (Leanna et al.), entitled MEDICAL DEVICES HAVING MICROPATTERN, filed on Mar. 15, 2013 (Atty. Docket No. 563.2-15934US01).

### BACKGROUND OF THE INVENTION

A wide variety of medical devices are known in the art. Some medical devices include a part that slidingly contacts another part of the medical device and/or biological tissue.

In some biopsy procedures it can be challenging to grab and biopsy enough tissue to analyze. Further, once the tissue has been biopsied it can be difficult to retrieve because the surface of the biopsy tool is slippery. Therefore, there remains a need in the art to provide a biopsy forceps to better retrieve and retain tissue material.

Without limiting the scope of the present disclosure, a brief summary of some of the claimed embodiments is set forth below. Additional details of the summarized embodiments of the present disclosure and/or additional embodiments of the present disclosure may be found in the Detailed Description of the Invention below. A brief abstract of the technical disclosure in the specification is also provided. The abstract is not intended to be used for interpreting the scope of the claims.

### BRIEF SUMMARY OF THE INVENTION

In some embodiments, a biopsy forceps system comprises a biopsy forceps having a first jaw and a second jaw. Each of the first jaw and second jaw has a cutting edge and a portion of at least one of the first jaw and the second jaw has a micropatterned coating thereon.

In some embodiments, the micropatterned coating comprises a polymeric material.

In some embodiments, the micropatterned coating is configured to increase the frictional coefficient of the portion of the at least one of the first jaw and the second jaw on which the micropatterned coating is located.

In some embodiments, the biopsy forceps further comprises a sliding surface having a micropatterned coating thereon.

In some embodiments, the micropatterned coating on the sliding surface is configured to reduce the frictional coefficient.

In some embodiments, the micropatterned coating on the sliding surface is lubricious.

In some embodiments, the biopsy forceps system further comprises a spike extending between the first and second jaws.

### BRIEF DESCRIPTION OF THE SEVERAL VIEWS OF THE DRAWING(S)

FIG. 1 shows an elevation view of an embodiment of a biopsy forceps.

FIG. 2 shows a side view of an embodiment of a biopsy forceps.

### DETAILED DESCRIPTION OF THE INVENTION

While the subject matter of the present disclosure may be embodied in many different forms, there are described in detail herein specific preferred embodiments of the present disclosure. This description is an exemplification of the principles of the present disclosure and is not intended to limit the present disclosure to the particular embodiments illustrated.

For the purposes of this disclosure, like reference numerals in the figures shall refer to like features unless otherwise indicated.

The present disclosure relates to employing micropatterned coatings for, for example, use on a biopsy forceps. With regard to FIG. 1, a biopsy forceps 10 is shown having jaws 12 which can be manipulated to take a tissue sample. In some embodiments, one or more of the jaws 12 includes a cavity 14 for retaining the tissue sample. With further regard to FIG. 1, the jaws 12 include a first jaw 12a and a second jaw 12b. The first jaw 12a is in opposing relationship with the second jaw 12b. The first and second jaws 12a, 12b each have a cutting edge 26, which cuts the tissue.

In some embodiments, one or more of the jaws 12 further includes one or more teeth 16, which can be located along the cutting edge 26. As shown in FIG. 1, for example, the teeth 16 on one of the jaws 12 can mate with the teeth 16 on an opposing jaw 12.

In some embodiments, the biopsy forceps 10 includes a micropatterned coating 18. In some embodiments, the micropatterned coating 18 extends over the entirety of the cavity 14. In some embodiments, the micropatterned coating 18 is disposed on one or more portions of the cavity 14. Further, in some embodiments, the micropatterned coating 18 is disposed on one or more teeth 16 and/or one or more portions of the teeth 16. In some embodiments, the micropatterned coating 18 is disposed on the outer surface 20 of the jaws 12. In some embodiments, the micropatterned coating 18 is disposed on one or more portions of the cutting edge 26.

In some embodiments, the micropatterned coating 18 helps to secure the tissue before taking the biopsy and further helps to retain the tissue as it is being removed from a body. In some embodiments, the micropatterned coating 18 provides extra friction to help the tissue remain engaged with the jaws 12 (e.g., metal jaws) while a biopsy technique is being performed. In some embodiments, the micropatterned coating 18 has a higher frictional coefficient than the surface of the biopsy forceps 10 on which it is applied or disposed. Suitable micropatterned coatings 18 are discussed, for example, in the Applications listed on page 1 of this Application.

In one or more embodiments, the micropatterned coating 18 comprises a micropatterned polymer. In some embodiments, the micropatterned coating 18 is applied on the outside of one or more of the jaws 12 to help with tangential and en face biopsies.

In addition to the foregoing, in some embodiments, the biopsy forceps 10 include a micropatterned coating 18 disposed on one or more sliding surfaces 22. The sliding surfaces 22 are, in some embodiments, adjacent surfaces that contact one another as the biopsy forceps 10 are actuated. In some embodiments, the micropatterned coating 18 disposed on the facing or sliding surface 22 is lubricious in order to reduce the frictional coefficient. This, in turn, can provide more control for the user, along with haptic feedback, and allow for more accurate tissue securement.

In one or more embodiments, the micropatterned coating 18 may provide a coated device or portion thereof with a non-stick characteristic, wherein articles that contact the non-stick surface may slide along the non-stick surface more easily and/or may be more cleanly removed from the non-stick surface. For example, in some embodiments, it may be desirable to include a lubricious micropatterned coating 18 on the outer surfaces of the biopsy forceps 10 and a friction inducing micropatterned coating 18 on the inside surfaces and teeth 16. Any suitable arrangement of friction enhancing and/or friction reducing micropatterned coating 18 can be used.

Further, in some embodiments, the micropatterned coating 18 is disposed on mating surfaces 30 of the teeth 16. In this way, when the mating surfaces 30 of the teeth 16 come together, they help retain the tissue sample.

Turning to FIG. 2, in some embodiments, the biopsy forceps 10 include a spike 24 disposed between the jaws 12. The spike 24 may be used to help anchor the biopsy forceps 10 in place before actuating the jaws 12 to obtain tissue. Anchoring the biopsy forceps 10 in place may be useful when, for example, slippery mucosa is present at or near the biopsy site. In one or more embodiments, a micropatterned coating 18 may be disposed on all or a portion of the spike 24 to increase the magnitude of frictional force between the spike 24 and the tissue.

It should be noted that the term “micropatterned coating”, as used herein, may refer to a separately manufactured polymer material that is applied to a surface or may refer to a polymer material that is manufactured simultaneously with the surface, or may refer to a medical device surface having a micropattern incorporated thereon. Further, micropatterned coatings may be formed of any suitable material for a particular application, and may include one or more of a flexible polymer, a rigid polymer, a metal, an alloy, and any other material that may be suitable for a particular application.

The entire contents of the following documents are herein incorporated by reference:

Lotters et al., “The mechanical properties of the rubber elastic polymer polydimethylsiloxane for sensor applications,” J. Micromech. Microengineering, 1997, 7(3): 145-147;

Axisa et al., “Low cost, biocompatible elastic and conformable electronic technologies using MID in stretchable polymer,” Conf. Proc. IEEE Eng. Med. Biol. Soc., 2007; 2007:6593-6;

Jeong et al., “Nanohairs and nanotubes: Efficient structural elements for gecko-inspired artificial dry adhesives,” Nano Today, August 2009, 4(4):335-346;

Majidi, “Enhanced Friction and Adhesion with Biologically Inspired Fiber Arrays,” University of California, Berkeley, Ph.D. thesis, May 15, 2007, 143 pgs.;

Mandavi et al., “A biodegradable and biocompatible gecko-inspired tissue adhesive,” Proc. Natl. Acad. Sci. U.S.A., 2008 Feb. 19; 105(7):2307-12;

Kroetch, “NanoFab's PDMS Microfluidic Device Fabrication Manual,” September 2004, 8 pgs. (available online at http://www.nanofab.ualberta.ca/wp-content/uploads/2009/03/boxedpdms.pdf, last accessed Mar. 10, 2013);

Dodou et al., “Mucoadhesive micropatterns for enhanced grip,” Conf. Proc. IEEE Eng. Med. Biol. Soc., 2007; 2007:1457-62;

Kwon et al., “Friction enhancement via micro-patterned wet elastomer adhesives on small intestinal surfaces,” Biomed. Mater., 2006 December; 1(4):216-20;

Tooley et al., “Thermal fracture of oxidized polydimethylsiloxane during soft lithography of nanopost arrays,” J. Micromech. Microeng., 2011, 21:054013 (9 pgs.);

Desai et al., “Plastic masters-rigid templates for soft lithography,” Lab Chip, 2009 Jun. 7; 9(11):1631-7).

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to.” Those familiar with the art may recognize other equivalents to the specific embodiments described herein which equivalents are also intended to be encompassed by the claims.

Further, the particular features presented in the dependent claims can be combined with each other in other manners within the scope of the present disclosure such that the present disclosure should be recognized as also specifically directed to other embodiments having any other possible combination of the features of the dependent claims.

This completes the description of the preferred and alternate embodiments of the present disclosure. Those skilled in the art may recognize other equivalents to the specific embodiment described herein which equivalents are intended to be encompassed by the claims attached hereto.

1. A biopsy forceps system comprising:
a biopsy forceps comprising a first jaw and a second jaw in opposing relationship with one another, wherein each of the first jaw and second jaw has a cutting edge;
wherein a portion of at least one of the first jaw and the second jaw has a micropatterned coating thereon.

2. The biopsy forceps of claim 1 wherein said first jaw includes a first cavity and said second jaw includes a second cavity, the micropatterned coating is disposed on one or more portions of the first cavity, the second cavity, or both.

3. The biopsy forceps of claim 2 wherein said micropatterned coating comprises a higher coefficient of friction than said first cavity or said second cavity.

4. The biopsy forceps of claim 1 wherein the micropatterned coating is disposed on one or more surfaces of the cutting edge of the first and second jaw.

5. The biopsy forceps of claim 4 wherein said first cutting edge comprises a first set of teeth and said second cutting edge comprises a second set of teeth, the first set of teeth is configured to mate with the second set of teeth, the micropatterned coating is disposed on one or more portions of the first and second set of teeth.

6. The biopsy forceps of claim 5 wherein said first set of teeth and said second set of teeth form mating surfaces, said micropatterned coating is disposed on said mating surfaces of the teeth.

7. The biopsy forceps system of claim 1, wherein the micropatterned coating comprises a polymeric material.

8. The biopsy forceps system of claim 1, wherein the micropatterned coating is configured to increase the frictional coefficient of the portion of the at least one of the first jaw and the second jaw on which the micropatterned coating is located.

9. The biopsy forceps system of claim 1, wherein the biopsy forceps further comprises a sliding surface having a micropatterned coating thereon.

10. The biopsy forceps system of claim 9, wherein the micropatterned coating on the sliding surface is configured to reduce the frictional coefficient.

11. The biopsy forceps system of claim 9, wherein the micropatterned coating on the sliding surface is lubricious.

12. The biopsy forceps system of claim 1 further comprising a spike extending between the first and second jaws.

13. The biopsy forceps system of claim 12 wherein said micropatterned coating is disposed on all or a portion of the spike.

14. The biopsy forceps system of claim 1 wherein said biopsy forceps comprises an outer surface, said micropatterned coating is disposed on all or a portion of the outer surface of the biopsy forceps.

15. The biopsy forceps system of claim 14 wherein said micropatterned coating is lubricious.

16. The biopsy forceps system of claim 14 wherein said micropatterned coating is a non-stick coating.

17. The biopsy forceps system of claim 14 wherein the first jaw comprises a first cavity and the second jaw comprises a second cavity, the micropatterned coating is provided on one or more portions of the first cavity and the second cavity.

18. The biopsy forceps system of claim 17 wherein the micropatterned coating is friction enhancing.

19. The biopsy forceps system of claim 17 wherein said first jaw comprises a first set of teeth and said second jaw comprises a second set of teeth configured to mate with the first set of teeth to form mating surfaces, at least a portion of the mating surfaces comprise the micropatterned coating.

20. The biopsy forceps system of claim 19 wherein said micropatterned coating comprises a friction enhancing material.
